(19) United States
(12) Patent Application Publication
(10) Pub. No.: US 2021/0142913 A1
(43) Pub. Date: May 13, 2021

(54) DIAGNOSIS SUPPORT DEVICE, DIAGNOSIS SUPPORT METHOD, AND NON-TRANSITORY RECORDING MEDIUM STORING DIAGNOSIS SUPPORT PROGRAM

(71) Applicant: OMRON HEALTHCARE Co., Ltd., Kyoto (JP)

(72) Inventors: Tadahisa TERAO, Kyoto (JP); Hirotaka WADA, Kyoto (JP); Tomohiro KUKITA, Kyoto (JP); Tamio UEDA, Kyoto (JP)

(21) Appl. No.: 17/151,342

(22) Filed: Jan. 18, 2021

Related U.S. Application Data
(63) Continuation of application No. PCT/JP2019/026081, filed on Jul. 1, 2019.

(30) Foreign Application Priority Data
Jul. 26, 2018 (JP) 2018-140353

(51) Int. Cl. G16H 50/30; G16H 10/20

(52) U.S. Cl. CPC G16H 50/30; G16H 10/20

(57) ABSTRACT

A diagnosis support device includes an acquisition unit configured to acquire health management information indicative of a health state or lifestyle pertaining to a patient, and medical interview item information including a plurality of question items associated with disease types, and a generation unit configured to generate medical interview sheet template data including question items corresponding to the health state or the lifestyle of the patient on the basis of the health management information and the medical interview item information that are acquired.

### CROSS-REFERENCE TO RELATED APPLICATIONS

This application is the U.S. national stage application filed pursuant to 35 U.S.C. 365(c) and 120 as a continuation of International Patent Application No. PCT/JP2019/026081, filed Jul. 1, 2019, which application claims priority from Japanese Patent Application No. 2018-140353, filed Jul. 26, 2018, which applications are incorporated herein by reference in their entireties.

### TECHNICAL FIELD

This invention relates to a diagnosis support device, a diagnosis support method, and a non-transitory recording medium storing a diagnosis support program that support medical personnel in diagnosing a patient.

### BACKGROUND ART

In Patent Document 1, a medical data management system that manages medical data for medical care and treatment is described. In this medical data management system, medical data of the past relating to patients is accumulated, and the accumulated medical data can be retrieved at an arbitrary terminal.

### CITATION LIST

### Patent Literature

Patent Document 1: JP 2002-366655 A

### SUMMARY OF INVENTION

### Technical Problem

There is a demand for the effective use of information relating to patients accumulated in medical data management systems in medical institutions, such as that described in Patent Document 1, for the diagnosis on patients.

The present invention has been made in light of the circumstances described above, and one aspect is directed at providing a diagnosis support device, a diagnosis support method, and a non-transitory recording medium storing a diagnosis support program capable of effectively using information relating to patients for diagnosis.

### Solution to Problem

In order to solve the problems described above, the present invention adopts the following measures, for example.

A diagnosis support device according to an embodiment of the present disclosure includes an acquisition unit configured to acquire health management information indicative of a health state or lifestyle pertaining to a patient, and medical interview item information including a plurality of question items associated with disease types, and a generation unit configured to generate medical interview sheet template data including question items corresponding to the health state or the lifestyle of the patient on the basis of the health management information and the medical interview item information that are acquired.

According to the configuration described above, when diagnosing a patient in a medical institution, the medical personnel, such as a physician, can use the medical interview screen generated on the basis of the current health state or lifestyle of the patient to perform a medical interview. By performing a diagnosis using the medical interview items corresponding to the current health state and lifestyle of the patient, the diagnosis can be performed efficiently and accurately.

In the diagnosis support device according to the embodiment described, the generation unit is configured to predict a disease type of the patient on the basis of the health management information being acquired, and generate medical interview sheet template data in which question items associated with the disease type being predicted are preferentially displayed.

In the diagnosis support device according to the embodiment described above, the generation unit is configured to predict a disease type of the patient on the basis of the health management information being acquired, select a question item that is associated with the disease type predicted from question items included in the medical interview item information, and generate medical interview sheet template data in which the question item being selected is displayed.

In the diagnosis support device according to the embodiment described above, the acquisition unit is configured to acquire, as the health management information, at least one of number of steps, amount of activity, travel history, weight, blood sugar level, diet, body temperature, or blood pressure.

In the diagnosis support device according to the embodiment described above, the acquisition unit is configured to further acquire medical information of the past pertaining to the patient, and the generation unit is configured to generate the medical interview sheet template data on the basis of the health management information, the medical information, and the medical interview item information that are acquired.

In the diagnosis support device according to embodiment described above, the acquisition unit is configured to acquire, as the medical information, at least of one of medical history information, preventive vaccination information, genetic information, medication information, or allergy information of the patient.

In the diagnosis support device according to the embodiment described above, the acquisition unit is configured to further acquire environment information of surroundings of the patient, and the generation unit is configured to generate the medical interview sheet template data on the basis of the health management information, diagnosis information, the medical interview item information, and the environment information that are acquired.

In the diagnosis support device according to the embodiment described above, as the environment information, at least of one of changes in air temperature, season, or epidemic information is acquired.

### Advantageous Effects of Invention

The present invention can provide a diagnosis support device, a diagnosis support method, and a non-transitory recording medium storing a diagnosis support program capable of effectively using information relating to patients for diagnosis.

### BRIEF DESCRIPTION OF DRAWINGS

FIG. 1 is a block diagram illustrating an example of the functional configuration of a diagnosis support device according to an application example.

FIG. 2 is a schematic diagram illustrating an example of the configuration of a diagnosis support system including a diagnostic support circuit, which is an example of a diagnosis support device according to a first embodiment.

FIG. 3 is a block diagram illustrating an example of the hardware configuration of a blood pressure measurement device according to the first embodiment.

FIG. 4 is a block diagram illustrating an example of the hardware configuration of a portable terminal according to the first embodiment.

FIG. 5 is a block diagram illustrating an example of the hardware configuration of a PHR server according to the first embodiment.

FIG. 6 is a block diagram illustrating an example of the hardware configuration of a hospital terminal according to the first embodiment.

FIG. 7 is a block diagram illustrating an example of the hardware configuration of a EMR server according to the first embodiment.

FIG. 8 is a block diagram illustrating an example of the functional configuration of the diagnostic support circuit, which is an example of a diagnosis support device according to the first embodiment.

FIG. 9 is a flowchart illustrating an example of the processing procedure of a medical interview data generation processing by the diagnosis support system including the diagnostic support circuit, which is an example of a diagnosis support device according to the first embodiment.

FIG. 10 is a diagram illustrating an example of a medical interview screen displayed on the basis of output data generated by the medical interview data generation processing of the diagnostic support circuit, which is an example of a diagnosis support device according to the first embodiment.

### DESCRIPTION OF EMBODIMENTS

Now, with reference to the drawings, embodiments are described. Note that, in the following description, constituent elements having the same function and configuration are denoted with a shared reference symbol. Further, when a plurality of constituent elements having a shared reference symbol are distinguished from one another, distinction is made by adding additional symbols following the shared reference symbol. Note that, when there is no particular need in distinguishing a plurality of constituent components, the plurality of constituent components are denoted only with a shared reference symbol without an additional symbol.

### 1. Application Example

First, with reference to FIG. 1, an example of a diagnosis support device to which the present invention is applied will be described.

A diagnosis support device 1 is used for medical interviews using a display device for a diagnosis subject by a medical personnel, for example, in a diagnosis at a medical institution such as a hospital. Medical personnel are, for example, physicians, nurses, and the like. The diagnosis subject is, for example, a patient. The display device is, for example, a portable terminal such as a tablet provided with a display screen. The diagnosis support device 1 executes medical interview screen generation processing on the basis of a diagnosis support program. As illustrated in FIG. 1, the diagnosis support device 1 includes an acquisition unit 2, a generation unit 3, and an output unit 4.

The acquisition unit 2 acquires PHR information and medical interview item information. In the present application example, the acquisition unit 2 further acquires EMR information.

Personal Health Records (PHR) is a medical and health record whereby individuals autonomously manage data obtained by the individual. The PHR information is information obtained from the PHR. The PHR information is indicative of the current health state or lifestyle of the patient. The PHR information is an example of health management information.

The PHR information may include biological information and behavior information. The biological information is information relating to a biological parameter of the patient. The biological information may include age, gender, weight, blood sugar level, body temperature, blood pressure information, heart rate, and the like. The blood pressure information is a systolic blood pressure, a diastolic blood pressure, or other index. The behavior information is information relating to behavior of the patient. The behavior information may include number of steps, amount of activity, travel information, dietary information, and the like.

Electronic Medical Records (EMR) is a medical and health record whereby data measured or diagnosed at a medical institution such as a hospital, for example, is managed. The EMR information is information relating to tests and diagnoses of the past at a medical institution. The EMR information is an example of medical information acquired at a medical institution.

The EMR information can include test information, diagnosis information, and the like. The test information includes height, weight, blood sugar level, genetic information, and the like. The diagnosis information may include medical history information, medication information, allergy information, preventive vaccination information, and the like.

The medical interview item information can include a plurality of question items. Question items are questions about the patient for identifying the disease type, the symptoms, the causes, and the like. Each of the question items is stored associated with one or more disease types with the highest correlation.

The generation unit 3 generates, for the patient of interest, a medical interview sheet template data corresponding to the current state and medical data of the past on the basis of the PHR information, the EMR information, and the medical interview item information. The medical interview sheet template data is display data for displaying the medical interview screen on the display screen of the display device. On the medical interview screen, a plurality of question items corresponding to the current state and medical data of the past of the patient of interest are displayed side by side. The generation unit 3 extracts one or more disease types, which the patient may have, on the basis of, for example, a table or a database indicating the relationship between each of the information included in the PHR information and the EMR information and the disease type. The generation unit 3 extracts one or more question items associated with the extracted disease type from among the plurality of question items included in the medical interview item information. Then, the generation unit 3 generates a medical interview sheet template data that displays the extracted question items.

In addition, the generation unit 3 sets the importance for each of the extracted question items. The generation unit 3, for example, gives the question items associated with the disease type, which the patient likely has, a higher importance than the question items associated with the disease type, which the patient likely does not have. Then, the generation unit 3 generates a medical interview sheet template data in which the extracted question items are arranged in order of importance from high to low.

The output unit 4 outputs, to the outside, the medical interview sheet template data generated by the generation unit 3.

According to the configuration described above, in diagnosing a patient at a medical institution, the medical interview can be performed using the medical interview screen generated on the basis of the PHR information. That is, a medical interview tailored to the patient's current health state and current lifestyle can be performed. Also, on the medical interview screen, question items relating to the disease type, which is determined that the patient likely has, are preferentially displayed. Thus, the person, such as a physician, performing the diagnosis, can preferentially and reliably obtain information about a disease type that the patient likely has, and thus advantages such as the efficiency of diagnosis and accuracy of diagnosis being improved and the burden on the patient during diagnosis being reduced can be expected.

Furthermore, according to the configuration described above, the EMR information is acquired in addition to the PHR information. Thus, a medical interview tailored to the patient's current health state, current lifestyle, medical data of the past, and the like can be performed. Thus, advantages such as the efficiency of diagnosis and accuracy of diagnosis being improved and the burden on the patient during diagnosis being reduced can be further expected.

The PHR information autonomously acquired by the individual is generally less reliable than the EMR information obtained by a medical institution. Thus, in addition to PHR information, a reliable diagnosis can be performed by generating a medical interview screen using the EMR information obtained by the medical institution.

### 2. First Embodiment

A first embodiment of the diagnosis support device according to the application example described above will be described below. The diagnosis support system described below includes an EMR server with a diagnostic support circuit. The diagnostic support circuit is an example of a diagnosis support device.

### 2.1 Overall Configuration Example

FIG. 2 is a diagram schematically illustrating an example of an application scenario of the diagnosis support system according to the present embodiment. In the example of FIG. 2, the diagnosis support system includes a hospital terminal 50 and an EMR server 70. The hospital terminal 50 and the EMR server 70 can be connected via a network NW, such as the Internet. The EMR is, for example, an electronic medical chart system. A plurality of the hospital terminals 50 may be provided. For communication between the hospital terminal 50 and the EMR server 70, near-field wireless communication or wired communication may be applied without going through the network NW.

In the example of FIG. 2, a diagnosis support system further includes a blood pressure measurement device 10, a portable terminal 30, and a PHR server 40. A plurality of each of the blood pressure measurement device 10 and the portable terminal 30 may be provided. The blood pressure measurement device 10 and the portable terminal 30 are connected via near-field wireless communication or wired communication. The portable terminal 30 can be connected to the PHR server 40 via the network NW. The portable terminal 30 may be further connected to the EMR server 70 via the network NW. Thus, the blood pressure measurement device 10 can be connected to the PHR server 40 and the EMR server 70 via the portable terminal 30. That is, the blood pressure measurement device 10 is capable of communicating with the PHR server 40 and the EMR server 70 via the portable terminal 30.

For example, the blood pressure measurement device 10 is a wearable device that can be worn at a desired measurement location (for example, a wrist). The blood pressure measurement device 10 is an example of a device in the possession of a patient. The blood pressure measurement device 10 measures a blood pressure value of the patient at a measurement location. The blood pressure measurement device 10 can transmit the blood pressure information including the measurement result of the blood pressure value and the like to the portable terminal 30. Furthermore, the blood pressure measurement device 10 can acquire the pulse information of the patient. The pulse information includes a pulse value and a pulse wave. The blood pressure measurement device 10 can transmit the pulse information to the portable terminal 30. The blood pressure measurement device 10 includes a clock function and can transmit the blood pressure information and the pulse information to the portable terminal 30 in association with the measured date/time.

For example, the portable terminal 30 is a terminal that can be carried by the patient. The portable terminal 30 is an example of a device in the possession of a patient. The portable terminal 30 receives the blood pressure information and the pulse information from the blood pressure measurement device 10. The portable terminal 30 can transfer the received blood pressure information and pulse information in association with the measured date/time to the PHR server 40. Additionally, in the portable terminal 30, the amount of activity, travel history, weight, blood sugar level, diet, body temperature, and the like are input as information autonomously managed by the patient. The portable terminal 30 can transfer the input information to the PHR server 40.

The PHR server 40 is a server computer that accumulates information transmitted from portable terminal 30. Personal Health Records (PHR) is a medical and health record whereby individuals autonomously manage data obtained by the individual. The PHR server 40 may transmit accumulated information to the EMR server 70.

The hospital terminal 50 is a terminal operable by a medical personnel, such as a physician, a nurse, and the like. The medical personnel consults with the patient and diagnoses a medical condition of the patient on the basis of test data and the like. The hospital terminal 50 can receive test data from a test device (not illustrated) or the like in a hospital, and present the test data to the medical personnel. In the hospital terminal 50, diagnosis information and the like relating to the patient are input. The hospital terminal 50 can transmit the diagnosis information to the EMR server 70. In addition, the hospital terminal 50 can receive the medical interview sheet template data from the EMR server 70 and display the medical interview screen based on the medical interview sheet template data. The hospital terminal 50 is an example of a display device.

The EMR server 70 is a server computer that accumulates information transmitted from the PHR server 40, each of the test devices, and the like. The EMR server 70 is an example of a server within a medical institution. Electronic Medical Records (EMR) is a medical and health record whereby data measured or diagnosed at a medical institution such as a hospital, for example, is managed. The accumulated information is stored as an electronic medical chart, for example. The EMR server 70 can transmit the stored information to the PHR server 40 and the hospital terminal 50.

The EMR server 70 includes a diagnostic support circuit 80. The diagnostic support circuit 80 obtains information relating to the patient accumulated in the EMR server 70 and generates the medical interview sheet template data based on the obtained information. The diagnostic support circuit 80 outputs the generated medical interview sheet template data as output data to the EMR server 70. The EMR server 70 can transmit the medical interview sheet template data generated by the diagnostic support circuit 80 to the PHR server 40 and the hospital terminal 50. The diagnostic support circuit 80 is an example of a diagnosis support device. The diagnostic support circuit 80 may be provided, for example, in the hospital terminal 50.

### 2.2 Hardware Configuration Examples

An example of the hardware configuration of each of the devices of the diagnosis support system according to the present embodiment will now be described.

### 2.2.1 Hardware Configuration Example of Blood Pressure Measurement Device

First, a hardware configuration example of the blood pressure measurement device 10 according to the present embodiment will be described. FIG. 3 is a block diagram illustrating an example of the hardware configuration of the blood pressure measurement device 10 according to the present embodiment. As illustrated in FIG. 3, the blood pressure measurement device 10 according to the present embodiment includes a control unit 11, a storage unit 12, a communication unit 13, an operation unit 14, a display unit 15, and a blood pressure sensor 16. The blood pressure measurement device 10 may further include at least one of an acceleration sensor 17 and a temperature/humidity sensor 18.

The control unit 11 includes a central processing unit (CPU), a random-access memory (RAM), a read-only memory (ROM), and the like, and controls each component in accordance with information processing. Further, the control unit 11 includes a clock (not illustrated), and has a function of acquiring a current date/time. The control unit 11 may have a function of displaying the acquired date/time on the display unit 15.

The control unit 11 generates blood pressure information, pulse information, activity information, and environment information on the basis of measurement results from the blood pressure sensor 16, the acceleration sensor 17, and the temperature/humidity sensor 18 and acquisition results from the network and the like. The blood pressure information includes, for example, a blood pressure value of the patient based on measurement by the blood pressure sensor 16. The pulse information includes, for example, a measurement result and a pulse value of the pulse wave of the patient based on measurement by the blood pressure sensor 16. The activity information includes the amount of activity, the number of steps, and a sleep condition of the patient based on measurement by the acceleration sensor 17. The environment information includes the temperature and the humidity around the patient based on measurements by the temperature/humidity sensor 18. The environment information also includes the current date, recent weather information, the current season, epidemic information, and the like. Each of the blood pressure information, the pulse information, the activity information, and the environment information is associated with the measured date/time based on the current date/time acquired by the clock. Further, each of the blood pressure information, the pulse information, the activity information, and the environment information may further be associated with a device ID for uniquely identifying the blood pressure measurement device 10.

The storage unit 12 is, for example, an auxiliary storage device such as a solid state drive, for example. In a case where the blood pressure measurement device 10 is configured as a somewhat large device rather than a small device such as a watch type, the storage unit 12 may be a hard disk drive. The storage unit 12 stores programs executed by the control unit 11, the blood pressure information, the pulse information, the activity information, the environment information, and the like.

The communication unit 13 is a communication interface for performing communication with the portable terminal 30. The communication unit 13 transmits, to the portable terminal 30, the blood pressure information, the pulse information, the activity information, the environment information, and the like, for example. In the present embodiment, for communication with the portable terminal 30 by the communication unit 13, near-field wireless communication such as Bluetooth (trade name), for example, can be applied, but the communication is not limited thereto. For example, communication performed by the communication unit 13 may adopt communication via the network NW such as a local area network (LAN) or wired communication through use of a communication cable.

For example, the operation unit 14 includes a user interface such as a touch panel and an operation button. The operation unit 14 detects an operation performed by the patient through the user interface, and outputs a signal indicative of the content of the detected operation to the control unit 11.

The display unit 15 includes, for example, a display screen, an indicator, and the like. The display screen includes, for example, a liquid crystal display (LCD), an electroluminescent (EL) display, or the like. The display unit 15 displays information in accordance with a signal from the control unit 11, and notifies the patient. The display unit 15 can display, for example, the blood pressure information, the pulse information, the activity information, the environment information, and the like stored in the storage unit 12.

The blood pressure sensor 16 measures the blood pressure value of the patient. The blood pressure value includes representative indices such as, for example, a systolic blood pressure and a diastolic blood pressure.

The blood pressure sensor 16 may be, for example, a continuous measurement type capable of measuring the blood pressure of the patient per beat of the heartbeat (continuously), or may be a non-continuous measurement type capable of measuring the blood pressure on the spot at a predetermined time (non-continuously). For example, the continuous measurement type blood pressure sensor 16 may adopt a method of measuring the blood pressure of the subject continuously on the basis of pulse transit time (PTT), a method of measuring the blood pressure continuously on the basis of a pressure pulse wave (tonometry method), or the like. Note that the method of measuring blood pressure continuously is not limited to the above-mentioned examples, and a method of detecting a pulse wave through use of a light emitting element and the like may be adopted as appropriate. For example, the non-continuous measurement type blood pressure sensor 16 may adopt a method of detecting a pulse wave by applying a pressure on a blood vessel through use of a cuff as a pressure sensor (oscillometric method). Thus, the blood pressure sensor 16 is capable of acquiring the pulse information.

The acceleration sensor 17 detects acceleration of the part of the patient on which the blood pressure measurement device 10 is worn, as a set of three axial components. Further, the acceleration sensor 17 may further includes a gyro sensor, and may further detect an angular velocity as a group of three-axial components in addition to acceleration.

The temperature/humidity sensor 18 measures the temperature and humidity around the patient.

### 2.2.2 Hardware Configuration Example of Portable Terminal

Next, a hardware configuration example of the portable terminal 30 will be described. FIG. 4 is a block diagram illustrating an example of the hardware configuration of the portable terminal 30 according to the present embodiment. As illustrated in FIG. 4, the portable terminal 30 according to the present embodiment includes a control unit 31, a storage unit 32, a communication unit 33, an operation unit 34, a display unit 35, and a global positioning system (GPS) receiver 36.

The control unit 31 and the storage unit 32 are similar to the control unit 11 and the storage unit 12 of the blood pressure measurement device 10, respectively. The storage unit 32 of the portable terminal 30, under the control of the control unit 31, stores the information received from the blood pressure measurement device 10 and position information generated by the GPS receiver 36. The information received from the blood pressure measurement device 10 includes the blood pressure information, the pulse information, the activity information, the environment information, and the like. Furthermore, the storage unit 32, under the control of the control unit 31, stores the biological information, the behavior information, and the like input at the operation unit 34. The biological information input at the operation unit 34 includes age, gender, weight, blood sugar level, body temperature, and the like. The behavior information input at the operation unit 34 includes travel information, dietary information, and the like. Note that, when the blood pressure information, the pulse information, the activity information, the environment information, the biological information, the behavior information, and the like are stored in the storage unit 32, the information can be stored together with an acquired date/time of the information. The travel information can also be generated on the basis of position information generated by the GPS receiver 36.

The communication unit 33 is a communication interface for performing communication with the blood pressure measurement device 10 and the PHR server 40. The communication unit 33 receives, for example, the blood pressure information, the pulse information, the activity information, the environment information, and the like from the blood pressure measurement device 10. Further, the communication unit 33 transmits the blood pressure information, the pulse information, the activity information, the environment information, the position information, the biological information, the behavior information, and the like to the PHR server 40.

The operation unit 34 and the display unit 35 are similar to the operation unit 14 and the display unit 15 of the blood pressure measurement device 10, respectively. The display unit 35 can display an input screen for the biological information and the behavior information. In the operation unit 34, the biological information, the behavior information, and the like can be input.

The GPS receiver 36 measures a position of the portable terminal 30, and generates the position information. For example, the position information includes a positioning date/time and a latitude and a longitude of the portable terminal 30 at the positioning date/time. For example, positioning performed by the GPS receiver 36 may be performed in synchronization with measurement performed by the blood pressure sensor 16 of the blood pressure measurement device 10.

### 2.2.3 Hardware Configuration Example of PHR Server

Next, a hardware configuration example of the PHR server 40 will be described. FIG. 5 is a block diagram illustrating an example of the hardware configuration of the PHR server 40 according to the present embodiment. As illustrated in FIG. 5, the PHR server 40 according to the present embodiment includes a control unit 41, a storage unit 42, and a communication unit 43.

The control unit 41 and the storage unit 42 are similar to the control unit 11 and the storage unit 12 of the blood pressure measurement device 10, respectively. The storage unit 42 of the PHR server 40 stores information transmitted from the portable terminal 30.

The communication unit 43 is a communication interface for performing communication with the portable terminal 30 and the EMR server 70. For example, the communication unit 43 receives, from the portable terminal 30, the blood pressure information, the pulse information, the activity information, the environment information, the biological information, the behavior information, and the like. The communication unit 43 can transmit information stored in the storage unit 42 to the portable terminal 30 and the EMR server 70.

### 2.2.4 Hardware Configuration Example of Hospital Terminal

Next, the hardware configuration example of the hospital terminal 50 will be described. FIG. 6 is a block diagram illustrating an example of the hardware configuration of the hospital terminal 50 according to the present embodiment. As illustrated in FIG. 6, the hospital terminal 50 according to the present embodiment includes a control unit 51, a storage unit 52, a communication unit 53, an operation unit 54, and a display unit 55.

The control unit 51 and the storage unit 52 are similar to the control unit 11 and the storage unit 12 of the blood pressure measurement device 10, respectively. The control unit 51 of the hospital terminal 50 generates the diagnosis information, the medication information, and the like relating to the patient.

The storage unit 52 of the hospital terminal 50 stores the diagnosis information, the medication information, and the like relating to the patient generated by the control unit 51. Further, the storage unit 52 stores the medical interview sheet template data and the like received from the EMR server 70.

The communication unit 53 is a communication interface for performing communication with the EMR server 70. The communication unit 53 transmits the diagnosis information, the medication information, and the like relating to the patient to the EMR server 70. Further, the communication unit 53 can receive the medical interview sheet template data and the like from the EMR server 70.

The operation unit 54 and the display unit 55 are similar to the operation unit 14 and the display unit 15 of the blood pressure measurement device 10, respectively. At the operation unit 54, the medication information and the test information acquired by various test devices, and the like can be input. The medication information and the test information may be recorded in the EMR server 70 via a network, such as a hospital LAN. In addition, the display unit 55 can display the medical interview screen based on the medical interview sheet template data generated by the diagnostic support circuit 80. The display unit 55 is an example of a display screen.

### 2.2.5 Hardware Configuration Example of EMR Server

Next, a hardware configuration example of the EMR server 70 will be described. FIG. 7 is a block diagram illustrating an example of the hardware configuration of the EMR server 70 according to the present embodiment. As illustrated in FIG. 7, the EMR server 70 according to the present embodiment includes a control unit 71, a storage unit 72, a communication unit 73, and the diagnostic support circuit 80.

The control unit 71 and the storage unit 72 are similar to the control unit 11 and the storage unit 12 of the blood pressure measurement device 10, respectively. The storage unit 72 of the EMR server 70 stores information transmitted from the PHR server 40, the hospital terminal 50, and each of the test devices. The information transmitted from the hospital terminal 50 and each of the test devices includes test information relating to test results, diagnosis information relating to diagnostic results, environment information, and the like. Also, the storage unit 72 of the EMR server 70 can also temporarily store the medical interview sheet template data obtained from the diagnostic support circuit 80.

The communication unit 73 is a communication interface for performing communication with the PHR server 40, the hospital terminal 50, and each of the test devices. For example, the communication unit 73 receives, from the PHR server 40, the blood pressure information, the pulse information, the activity information, the environment information, the biological information, the behavior information, and the like. In addition, the communication unit 73 receives the test information, the diagnosis information, and the environment information from the hospital terminal 50, each of the test devices, and the like. Note that the communication unit 73 may receive the environment information acquired by the portable terminal 30 and the like from the PHR server 40 and the like. The communication unit 73 can transmit information stored in the storage unit 72 to the PHR server 40 and the hospital terminal 50.

The diagnostic support circuit 80 includes a processor 80a and a memory 80b, for example. The diagnostic support circuit 80 implements various operation controls, data processing, and the like by the processor 80a executing programs stored in the memory 80b. Further, the diagnostic support circuit 80 can include a clock (not illustrated) to clock the current date/time.

The processor 80a is, for example, a CPU or a micro processing unit (MPU) including an arithmetic circuit, or the like. The processor 80a is capable of executing the control and data processing of each unit by executing a program stored in the memory 80b or the storage unit 72.

The memory 80b includes, for example, a non-volatile memory that stores programs to be executed by the processor 80a, and a volatile memory, such as a RAM, for use as a working memory.

The diagnostic support circuit 80 executes medical interview data generation processing on the basis of a medical interview data generation program. The medical interview data generation program is an example of a non-transitory recording medium storing a diagnosis support program. The medical interview data generation processing by the diagnostic support circuit 80 will be described below. The medical interview data generation program is a program for causing the diagnostic support circuit 80 to execute the medical interview data generation processing. The medical interview data generation program may be stored in the memory 80b or may be stored in the storage unit 72.

Note that the control unit 71 may function as the diagnostic support circuit 80. That is, the control unit 71 may also be used as the diagnostic support circuit 80. In this case, the CPU of the control unit 71 serves as the processor 80a of the diagnostic support circuit 80, the ROM of the control unit 71 serves as the non-volatile memory of the memory 80b of the diagnostic support circuit 80, and the RAM of the control unit 71 serves as the volatile memory of the memory 80b of the diagnostic support circuit 80.

### 2.3 Functional Configuration Examples

Next, an example of the functional configuration of the diagnosis support system according to the present embodiment will be described.

### 2.3.1 Functional Configuration Example of Diagnostic Support Circuit

FIG. 8 is a block diagram schematically illustrating an example of the functional configuration of the diagnostic support circuit 80 of the diagnosis support system according to the present embodiment.

The processor 80a of the diagnostic support circuit 80 deploys the medical interview data generation program stored in the non-volatile memory of the memory 80b in the volatile memory of the memory 80b. The processor 80a then interprets and executes the medical interview data generation program deployed in the volatile memory, thereby functioning as an acquisition unit 81, a generation unit 82, and an output unit 83.

The volatile memory of the memory 80b functions as a PHR information storage unit 86a, an EMR information storage unit 86b, a diagnosis-use information storage unit 86c, an environment information storage unit 86d, and an output data storage unit 86e.

The PHR information storage unit 86a stores PHR information. The PHR information is information acquired from the blood pressure measurement device 10 and the portable terminal 30 via the PHR server 40. The blood pressure measurement device 10 and the portable terminal 30 are examples of a device in the possession of a patient. The PHR information is indicative of the current health state or lifestyle of the patient. The PHR information is an example of health management information.

The PHR information may include biological information and behavior information. The biological information is information relating to a biological parameter of the patient. The biological information may include age, gender, weight, blood sugar level, body temperature, blood pressure information, heart rate, and the like. The blood pressure information is a systolic blood pressure, a diastolic blood pressure, or other index. The behavior information is information relating to behavior of the patient. The behavior information may include number of steps, amount of activity, travel information, dietary information, and the like.

The EMR information storage unit 86b stores EMR information. The EMR information is information relating to tests and diagnoses of the past at a medical institution. The EMR information is information acquired from the EMR server 70. The EMR server 70 is an example of a server within a medical institution. The EMR information is an example of medical information acquired at a medical institution.

The EMR information can include test information, diagnosis information, and the like. The test information may include height, weight, blood sugar level, genetic information, and the like. The diagnosis information may include medical history information, medication information, allergy information, preventive vaccination information, and the like.

The diagnosis-use information storage unit 86c stores diagnosis-use information. The diagnosis-use information may include medical interview item information. The medical interview item information can include a plurality of question items. Question items are questions for identifying the disease type, the symptoms, the causes, and the like. Each of the question items is stored associated with one or more disease types with the highest correlation. The diagnosis-use information may further include a table or a database indicating the relationship between the PHR information and the EMR information and the disease type the patient likely has. Also, the diagnosis-use information may further include a table or a database indicating the relationship between the environment information and the disease type the patient may have.

The environment information storage unit 86d stores environment information. The environmental information is information relating to the patient's surroundings. The environmental information relates to the current time and environment. The environmental information may include the current date, recent weather information, the current season, epidemic information, and the like. The epidemic information is information relating to the current epidemic of diseases. Recent weather information can include, for example, recent changes in air temperature and the like.

The output data storage unit 86e stores the medical interview sheet template data as output data. The medical interview sheet template data is image data for displaying the medical interview screen on the display screen of the display device. The medical interview sheet template data may be image data for printing for displaying medical interview items on a printing medium. On the medical interview screen, a plurality of question items are displayed side by side.

Note that, while an example has been described in which the information of each patient is stored in the respective information storage units, the present invention is not limited thereto. For example, a plurality of storage units may be provided for each patient.

The acquisition unit 81 acquires PHR information, EMR information, diagnosis-use information, and environment information. The acquisition unit 81 acquires the pieces of information from the communication unit 73 or the storage unit 72, for example. The acquisition unit 81 stores acquired pieces of information in respective regions of the storage units 86a to 86d of the memory 80b.

The generation unit 82 generates the medical interview sheet template data corresponding to the health state or the lifestyle of the patient of interest on the basis of, from among the pieces of information stored in the memory 80b, at least the PHR information, the EMR information, and the diagnosis-use information. In the present embodiment, the generation unit 82 generates the medical interview sheet template data corresponding to the patient of interest on the basis of the PHR information, the EMR information, the diagnosis-use information, and the environment information stored in the memory 80b. The display device is, for example, the hospital terminal 50, and the display screen is, for example, the display unit 55. The generation unit 82 stores the generated medical interview sheet template data in the output data storage unit 86e of the memory 80b.

In the process of generating the medical interview sheet template data, the generation unit 82 first predicts a disease type, which the patient may have, on the basis of the PHR information, the EMR information, the diagnosis-use information, and the environment information stored in the memory 80b. At this time, the generation unit 82 extracts one or more disease types, which the patient may have, on the basis of, for example, a table or a database indicating the relationship between the pieces of information included in the PHR information and the EMR information and the disease type that the patient may have. The generation unit 82 extracts one or more question items associated with the extracted disease type from among the plurality of question items included in the medical interview item information. Then, the generation unit 82 generates a medical interview sheet template data that displays the extracted question items. Also, the generation unit 82 can predict the disease type which the patient may have, on the basis of a table or a database indicating the relationship between the environment information and the disease type that the patient may have, and on the basis of the environment information.

In addition, the generation unit 82 sets the importance for each of the extracted question items. The generation unit 82, for example, gives the question items associated with the disease type, which the patient likely has, a higher importance than the question items associated with the disease type, which the patient likely does not have. Then, the generation unit 82 may generate a medical interview sheet template data in which the extracted question items are arranged in order of importance from high to low.

The output unit 83 outputs the medical interview sheet template data stored in the output data storage unit 86e of the memory 80b as output data to the storage unit 72 of the EMR server 70.

### 2.4 Operation Examples

Next, an operation example of the diagnosis support system according to the present embodiment will be described. Note that the processing procedure described below is merely an example, and each process may be changed to the extent possible. Further, in the processing procedure described below, steps can be omitted, substituted, and added in accordance with the embodiment as appropriate.

FIG. 9 is a flowchart illustrating an example of the process of the medical interview data generation processing by the processor 80a of the diagnostic support circuit 80 according to the present embodiment. The processor 80a starts the medical interview data generation processing on the basis of, for example, an operation input to display the medical interview screen on the display unit 55 of the hospital terminal 50.

In the medical interview data generation processing, the processor 80a first obtains PHR information, EMR information, diagnosis-use information, and environment information from, for example, the storage unit 72 of the EMR server 70 (step S101). At this time, the processor 80a acquires the PHR information and the EMR information relating to the patient to be diagnosed.

Next, the processor 80a predicts the disease type, which the patient may have, on the basis of the PHR information, the EMR information, the diagnosis-use information, and the environment information that are acquired by the processing of step S101 (step S102). The processor 80a extracts one or more disease types, which the patient may have, on the basis of, for example, a table or a database indicating the relationship between the PHR information and the EMR information and the disease type that the patient may have.

Next, the processor 80a extracts a question item associated with the predicted disease type from among the question items of the medical interview item information on the basis of the disease type predicted by the processing of step S102 (step S103). For each of the disease types predicted in the processing of step S102, the processor 80a, for example, extracts one or more question items relating to symptoms that develop in case of having the disease of the predicted disease type.

Next, processor 80a sets the importance for each of the extracted question items (step S104). The processor 80a, for example, gives the question items associated with the disease type, which the patient likely has, a higher importance than the question items associated with the disease type, which the patient likely does not have.

Next, the processor 80a generates the medical interview sheet template data on the basis of the extracted question item (step S105). At this time, the processor 80a generates the medical interview sheet template data with the question items displayed arranged in order of importance from high to low on the basis of importance set for each of the question items.

Next, the processor 80a outputs the generated medical interview sheet template data as output data to outside (step S106). The processor 80a outputs the medical interview sheet template data to the control unit 71 of the EMR server 70, for example.

The medical interview sheet template data output to the EMR server 70 is transmitted by the EMR server 70 to the hospital terminal 50. Then, on the hospital terminal 50, a medical interview screen is displayed on the display unit 55 on the basis of the received medical interview sheet template data.

### 2.5 Display Example

FIG. 10 illustrates a display example of the medical interview screen generated by the medical interview screen generation processing of the diagnostic support circuit 80. A medical interview display screen 90 is an example of a medical interview screen. The medical interview display screen 90 is displayed on the display unit 55 of the hospital terminal 50, for example, and is used in a medical interview of the diagnosis subject by the medical personnel. Medical personnel are, for example, physicians, nurses, and the like. The diagnosis subject is, for example, a patient. The medical interview display screen 90 includes a personal information display unit 91 and a question display unit 92.

On the personal information display unit 91, the personal information is displayed. The personal information is information for identifying an individual. The personal information may include a management number, gender, age, and the like. The personal information may further include a patient name and the like. In addition to personal information, the personal information display unit 91 may display PHR information.

In the question display unit 92, a plurality of question items and an answer field corresponding to each of the question items are displayed. In the question display unit 92, the question items relating to the disease type, predicted on the basis of the PHR information, EMR information, and the environment information, are displayed. Also, in the question display unit 92, the question items are displayed in order of importance from high to low, for example.

For example, when it is determined that the patient likely has a cold on the basis of the body temperature and pulse of the patient and recent changes in air temperature, question items relating to symptoms of a cold and causes of a cold are preferentially displayed on the question display unit 92 of the medical interview display screen 90. In this case, on the question display unit 92, for example, question items relating to the presence or absence of a headache, abdominal pain, cough, and the like; recent sleeping status; and recent dietary status are preferentially displayed.

Also, for example, regarding the patient, when the body temperature on the day is equal to or greater than normal temperature by a predetermined temperature and influenza is prevalent, it is determined that the patient likely has influenza, and question items relating to influenza are preferentially displayed on the question display unit 92. The question items relating to influenza are, for example, questions relating to the presence or absence of symptoms commonly developed in patients with influenza. At this time, for example, question items relating to influenza are placed on the upper side of the medical interview screen and other question items are placed on the lower side of the medical interview screen. In this way, for example, even when an answer to a question item placed on the lower side of the medical interview screen is not obtained, because the question items relating to the predicted disease type are preferentially displayed, a diagnosis of a disease type the patient likely has can be performed with high probability, and highly efficient diagnoses can be expected.

Also, the disease type, which the patient currently has, is determined not likely to be a disease type for which preventive vaccination has been received on the basis of information relating to preventive vaccination. In this case, in the question display unit 92, the importance of the question items, relating to the symptoms of the disease type determined not likely, is determined to be low, and the question items relating to other disease types are preferentially displayed.

### 2.6 Actions and Effects

Next, actions and effects of the diagnosis support system according to the present embodiment will be described.

In the present embodiment, in diagnosing to a patient a medical institution, the medical interview can be performed using the medical interview screen generated on the basis of the PHR information, the EMR information, and the environment information. That is, a medical interview tailored to the patient's current health state, current lifestyle, medical data of the past, and the like can be performed. Also, on the medical interview screen, question items relating to the disease type that patient likely has are preferentially displayed. Thus, the person, such as a physician, performing the diagnosis, can preferentially and reliably obtain information about a disease type that the patient likely has, and thus advantages such as the efficiency of diagnosis and accuracy of diagnosis being improved and the burden on the patient during diagnosis being reduced can be expected.

The PHR information autonomously acquired by the individual is generally less reliable than the EMR information obtained by a medical institution. In this embodiment, in addition to the PHR information, the EMR information obtained at the medical institution can be used to generate the medical interview screen basing on a more reliable prediction.

### 3. Common Configurations of Embodiments and the Like

A diagnosis support device (1, 80) includes an acquisition unit (2, 81) configured to acquire health management information indicative of a health state or lifestyle pertaining to a patient, and medical interview item information including a plurality of question items associated with disease types, and a generation unit (3, 82) configured to generate medical interview sheet template data including question items corresponding to the health state or the lifestyle of the patient on the basis of the health management information and the medical interview item information that are acquired.

Note that the present invention is not limited to the embodiment, and various modifications can be made in an implementation stage without departing from the gist. Further, embodiments may be carried out as appropriate in a combination, and combined effects can be obtained in such a case. Further, the various inventions are included in the embodiment, and the various inventions may be extracted in accordance with combinations selected from the plurality of disclosed constituent elements. For example, in a case where the problem can be solved and the effects can be obtained even when some constituent elements are removed from the entire constituent elements given in the embodiment, the configuration obtained by removing the constituent elements may be extracted as an invention.

### Supplementary Notes

A part or the entirety of the embodiment can be described as described in the following supplementary notes in addition to the scope of the claims, but the present invention is not limited thereto.

### (Supplementary Note 1)

A diagnosis support device (1, 80) including a hardware processor (80a) and a memory (80b), wherein
the hardware processor (80a) acquires health management information indicative of a health state or lifestyle pertaining to a patient and medical interview item information including a plurality of question items associated with disease types, and stores, in the memory (80b), the health management information and the medical interview item information being acquired; and
generates medical interview sheet template data including question items corresponding to the health state or the lifestyle of the patient on the basis of the health management information and the medical interview item information stored in the memory (80b).

### REFERENCE SIGNS LIST

1 Diagnosis support device

2 Acquisition unit

3 Generation unit

4 Output unit

10 Blood pressure measurement device

11 Control unit

12 Storage unit

13 Communication unit

14 Operation unit

15 Display unit

16 Blood pressure sensor

17 Acceleration sensor

18 Temperature/humidity sensor

30 Portable terminal

31 Control unit

32 Storage unit

33 Communication unit

34 Operation unit

35 Display unit

36 GPS receiver

40 PHR Server

41 Control unit

42 Storage unit

43 Communication unit

50 Hospital terminal

51 Control unit

52 Storage unit

53 Communication unit

54 Operation unit

55 Display unit

70 EMR server

71 Control unit

72 Storage unit

73 Communication unit

80 Diagnostic support circuit

80a Processor

80b Memory

81 Acquisition unit

82 Generation unit

83 Output unit

86a PHR information storage unit

86b EMR information storage unit

86c Diagnosis-use information storage unit

86d Environment information storage unit

86e Output data storage unit

90 Medical interview display screen

91 Personal information display unit

92 Question display unit

1. A diagnosis support device, comprising:
a processor configured to:
acquire health management information indicative of a health state or lifestyle pertaining to a patient, and medical interview item information including a plurality of question items associated with disease types; and
generate medical interview sheet template data including question items corresponding to the health state or the lifestyle of the patient on the basis of the health management information and the medical interview item information that are acquired,
wherein the processor is further configured to:
predict a disease type of the patient on the basis of the health management information being acquired,
set an importance to each one of the plurality of question items on the basis of the disease type being predicted, making an importance of a question item associated with a disease type that the patient likely has, higher than an importance of a question item associated with a disease type that the patient likely does not have, and
generate medical interview sheet template data in that the plurality of question items are arranged in order of importance from high to low.

2. The diagnosis support device according to claim 1, wherein
the processor is further configured to predict a disease type of the patient on the basis of the health management information being acquired, select a question item that is associated with the disease type predicted from question items included in the medical interview item information, and generate medical interview sheet template data in which the question item being selected is displayed.

3. The diagnosis support device according to claim 1, wherein
the processor is further configured to acquire, as the health management information, at least one of number of steps, amount of activity, travel history, weight, blood sugar level, diet, body temperature, or blood pressure.

4. The diagnosis support device according to claim 1, wherein
the processor is further configured to:
further acquire medical information of the past pertaining to the patient; and
generate the medical interview sheet template data on the basis of the health management information, the medical information, and the medical interview item information that are acquired.

5. The diagnosis support device according to claim 4, wherein
the processor is further configured to acquire, as the medical information, at least of one of medical history information, preventive vaccination information, genetic information, medication information, or allergy information of the patient.

6. The diagnosis support device according to claim 1, wherein
the processor is further configured to:
further acquire environment information of surroundings of the patient; and
generate the medical interview sheet template data on the basis of the health management information, the medical interview item information, and the environment information that are acquired.

7. The diagnosis support device according to claim 6, wherein
the processor is further configured to acquire, as the environment information, at least of one of changes in air temperature, season, or epidemic information.

8. A non-transitory recording medium storing a diagnosis support program for causing the processor to execute functions provided in the diagnosis support device according to claim 1.

9. A diagnosis support method executed by a diagnosis support device, comprising:
acquiring health management information indicative of a health state or lifestyle pertaining to a patient, and medical interview item information including a plurality of question items associated with disease types; and
generating medical interview sheet template data including question items corresponding to the health state or the lifestyle of the patient on the basis of the health management information and the medical interview item information that are acquired, wherein
the generating medical interview sheet template data includes,
predicting a disease type of the patient on the basis of the health management information being acquired,
setting an importance to each one of the plurality of question items on the basis of the disease type being predicted, making an importance of a question item associated with a disease type that the patient likely has, higher than an importance of a question item associated with a disease type that the patient likely does not have, and
generating medical interview sheet template data in that the plurality of question items are arranged in order of importance from high to low.

10. A non-transitory recording medium storing a diagnosis support program for causing the processor to execute functions provided in the diagnosis support device according to claim 2.

11. A non-transitory recording medium storing a diagnosis support program for causing the processor to execute functions provided in the diagnosis support device according to claim 3.

12. A non-transitory recording medium storing a diagnosis support program for causing the processor to execute functions provided in the diagnosis support device according to claim 4.

13. A non-transitory recording medium storing a diagnosis support program for causing the processor to execute functions provided in the diagnosis support device according to claim 5.

14. A non-transitory recording medium storing a diagnosis support program for causing the processor to execute functions provided in the diagnosis support device according to claim 6.

15. A non-transitory recording medium storing a diagnosis support program for causing the processor to execute functions of provided in the diagnosis support device according to claim 7.
